(19) United States
(12) Patent Application Publication
(10) Pub. No.: US 2010/0015051 A1
(43) Pub. Date: Jan. 21, 2010

(54) Transferrin-conjugated nanoparticles for increasing efficacy of a therapeutic agent

(76) Inventors: Vinod D. Labhasetwar, Omaha, NE (US); Sanjeeb K. Sahoo, Omaha, NE (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/124,934

(22) Filed: May 9, 2005

Related U.S. Application Data
(63) Continuation-in-part of application No. 10/955,739, filed on Sep. 30, 2004, now Pat. No. 7,332,159.

(51) Int. Cl. A61K 51/08; A61P 35/00

(52) U.S. Cl. 424/1.69

(57) ABSTRACT

The present invention relates to a transferrin-conjugated nanoparticle composition with sustained release characteristics. A method for increasing the efficacy of therapeutic agents and a method for treating cancer using the transferrin-conjugated nanoparticle composition are also provided.

This application is a Continuation-in-Part Application of U.S. patent application Ser. No. 10/955,739, filed Sep. 30, 2004, the content of which is incorporated by reference herein in its entirety.

### BACKGROUND OF THE INVENTION

The primary function of transferrin, a glycoprotein found abundantly in the blood, is to transport iron through the blood to cells through the transferrin receptors (Andrews (2000) Nat. Rev. Genet. 1:208-217; Qian and Tang (1995) Biochim. Biophys. Acta 1269:205-214). Since transferrin receptors are over-expressed in malignant tissues, transferrin has been extensively investigated as a ligand for targeting of antineoplastic agents (Qian, et al. (2002) Pharmacol. Rev. 54:561-587; Widera, et al. (2003) Adv. Drug. Deliv. Rev. 55:1439-1466). Further, transferrin receptors are over-expressed in certain body tissue such as in the liver, epidermis, intestinal epithelium, vascular endothelium of the brain capillary, and certain populations of blood cells in the bone marrow (Li and Qian (2002) Med. Res. Rev. 22:225-250; Li, et al. (2002) Trends Pharmacol. Sci. 23:206-209). Brain delivery of therapeutic agents using transferrin ligand has been used to facilitate the transcytosis of conjugated drug carrier systems across the blood-brain barrier, which is otherwise impermeable to most therapeutic agents (Pardridge (2002) Adv. Exp. Med. Biol. 513:397-430; Bickel, et al. (2001) Adv. Drug Deliv. Rev. 46:247-279).

Targeting approaches via transferrin ligand have involved conjugating drug molecules themselves or drug-loaded nanosystems such as liposomes, micelles and polymer-drug complexes (Langer (1998) Nature 392:5-10; Maruyama, et al. (1999) Adv. Drug Deliv. Rev. 40:89-102; Vyas, et al. (2001) Crit. Rev. Ther. Drug Carrier Syst. 18:1-76). Further, U.S. Pat. No. 6,254,890 discloses biodegradable nanosphere systems having a transferrin targeting moiety attached to the surface and methods for using the same to transport and release therapeutic agents, specifically nucleic acids. This patent teaches a system for the biocompatible transport and release of nucleic acids comprising nanospheres containing nucleic acids, wherein the nanospheres are sub-150 nm polymer spheres of which at least 50% of the size distribution of spheres is sub-100 nm. The biodegradable nanosphere can be composed of poly (e-caprolactone, poly (hydroxybutyrate), or poly (orthoesters). Moreover, WO 03/088950 discloses a method and compositions for targeted drug delivery. The compositions include a targeting molecule that specifically binds to a receptor on the surface of the targeted cells (e.g., hormone receptors or transferrin receptors); a drug to be delivered, such as a therapeutic, prophylactic, or diagnostic agent; and a nanoparticle, which contains on or within the nanoparticle, the drug to be delivered, as well as has attached thereto, the targeting molecule. Nanoparticles can consist of drug or drug associated with carrier, such as a controlled or sustained release materials like a poly(lactide-co-glycolide), a liposome or surfactant. Other suitable polymers include polyamides, polycarbonates, polyvinyl alcohols, polyvinyl ethers, or copolymers or blends thereof.

Biodegradable nanoparticles have been used as a drug delivery system for antineoplastic agents. Biodegradable nanoparticles provide sustained drug release and limit exposure of the drug to the cell membrane-associated efflux transporters (Brigger, et al. (2002) Adv. Drug Deliv. Rev. 54:631-651; Panyam and Labhasetwar (2003) Adv. Drug Deliv. Rev. 55:329-34; Sahoo and Labhasetwar (2003) Drug Discov. Today 8:1112-1120). Further, the slow intracellular release of drug from the nanoparticles localized inside cells is expected to sustain the drug effect and therefore can increase its overall efficacy (Panyam and Labhasetwar (2003) supra; Panyam, et al. (2002) FASEB J. 16:1217-1226). However, it has been found that a major fraction of the internalized nanoparticles undergoes rapid exocytosis (Panyam and Labhasetwar (2003) Pharm. Res. 20:212-220). This seems to occur because a large fraction of the internalized nanoparticles remains in the recycling endosomes, which subsequently undergo exocytosis.

Paclitaxel is a chemotherapeutic agent with a wide spectrum of antitumor activity. However, its therapeutic application in cancer therapy is limited, in part, due to its low water solubility which necessitates the use of a CREMOPHOR® EL formulation. In addition to the hypersensitivity reactions to CREMOPHOR® EL, the multiple dosing of the drug, required to maintain the therapeutic drug concentration in the tumor, causes nonspecific toxicity (Gelderblom, et al. (2002) Clin. Cancer Res. 8:1237-41; Weiss, et al. (1990) J. Clin. Oncol. 8:1263-8). Polymeric-, micellar-, and liposome-based delivery systems conjugated to tumor-specific ligands have been investigated to increase efficacy (Qian, et al. (2002) supra; Sapra and Allen (2003) Prog. Lipid Res. 42:439-62; Singh (1999) Curr. Pharmacol. Des. 5:443-51).

There is a need in the art for a biodegradable, sustained-release drug delivery system which targets a therapeutic agent, such as paclitaxel, to a cell and increases intracellular retention and efficacy of the encapsulated therapeutic agent. The present invention meets this long-felt need.

### SUMMARY OF THE INVENTION

The present invention is a composition for sustained-release of a therapeutic agent. The composition is a therapeutic agent encapsulated in a nanoparticle composed of at least one biodegradable polymer, a functional group, and a transferrin ligand conjugated to the functional group. In one embodiment, the composition further contains a radionuclide bound to the transferrin ligand.

The present invention is also a method for increasing the efficacy of a therapeutic agent by administering the therapeutic agent, in a transferrin-conjugated nanoparticle, to a subject in need of treatment with the therapeutic agent.

The present invention is further a method for treating cancer by administering to a subject, having or at risk of having cancer, an effective amount of a cancer therapeutic encapsulated in a biodegradable nanoparticle, wherein said nanoparticle has a transferrin ligand conjugated to the surface thereof.

### BRIEF DESCRIPTION OF THE DRAWINGS

FIG. 1 shows exocytosis of transferrin-conjugated and unconjugated nanoparticles in MCF-7 cells. Cells were incubated with nanoparticles (open bars) or transferrin-conjugated nanoparticles (filled bars) (100 μg/mL) for 1 hour, washed, and incubated with fresh medium. Nanoparticle levels were taken as cellular uptake (0 hour time point). Cells after washing were incubated with medium in other wells and were processed as above at different time points following washing to determine nanoparticle retention.

FIG. 2 shows intracellular paclitaxel levels following treatment with tritiated-paclitaxel either in solution (squares) or encapsulated in unconjugated nanoparticles (diamonds) or transferrin-conjugated nanoparticles (triangles) at paclitaxel concentrations of 10 ng/mL. The medium was changed at two days and then on every alternate day thereafter with no further dose of the drug added. At different time points, cells were washed and radioactivity was measured by using a liquid scintillation counter. Data are means±s.e.m (n=3), *p<0.05 paclitaxel-encapsulated, transferrin-conjugated nanoparticles or unconjugated paclitaxel-encapsulated nanoparticles versus paclitaxel in solution, **p<0.005 paclitaxel-encapsulated transferrin-conjugated nanoparticles or unconjugated paclitaxel-encapsulated nanoparticles versus paclitaxel in solution.

FIG. 3 shows dose- and time-dependent cytotoxicity of paclitaxel in MCF-7 (FIG. 3A and FIG. 3B) and MCF-7/Adr (FIG. 3C and FIG. 3D) cells. Different concentrations of paclitaxel either in solution (diamonds) or encapsulated in unconjugated nanoparticles (squares) or transferrin-conjugated nanoparticles (triangles) were added to wells with medium or nanoparticles (without drug) or transferrin-conjugated nanoparticles (without drug) acting as respective controls for drug in solution or paclitaxel-loaded nanoparticles or transferrin-conjugated paclitaxel-loaded nanoparticles. The medium was changed at two days and then on every alternate day thereafter with no further dose of the drug added. The extent of growth inhibition was measured at 5 days (FIG. 3A and FIG. 3C). The extent of growth inhibition with incubation time was measured at 1 ng/mL in MCF-7 (FIG. 3C) and at 1000 ng/mL in MCF-7/Adr cells (FIG. 3D) at 2, 5 and 8 days following treatment. The inhibition was calculated with respect to the respective controls, i.e., drug in solution with medium control and unconjugated, paclitaxel-encapsulated nanoparticles with control nanoparticles. Data as mean±s.e.m., n=6, *p<0.005 paclitaxel-encapsulated, transferrin-conjugated nanoparticles versus paclitaxel in solution and unconjugated paclitaxel-encapsulated nanoparticles.

### DETAILED DESCRIPTION OF THE INVENTION

The present invention relates to use of biodegradable, transferrin-conjugated nanoparticles containing a therapeutic agent for increased intracellular retention and efficacy of the therapeutic agent. To illustrate the efficacy of the inventive transferrin-conjugated nanoparticles, paclitaxel was encapsulated and used as a model antineoplastic agent in breast and prostate cancer. It was found that the inventive nanoparticle formulations provided greater antitumor activity of the therapeutic agent upon conjugation to transferrin ligand via an epoxy chain linker.

Accordingly, the present invention is a sustained-release, biodegradable nanoparticle composition for delivery of a therapeutic agent. As used herein, controlled release, sustained release, or similar terms are used to denote a mode of therapeutic agent delivery that occurs when the therapeutic agent is released from the nanoparticle formulation at an ascertainable and controllable rate over a period of time, rather than dispersed immediately upon application or injection. Controlled or sustained-release can extend for hours, days or months, and can vary as a function of numerous factors. For the composition of the present invention, the rate of release will depend on the rate of hydrolysis of the linkages between and within the polymers of the nanoparticle, particle size, acidity of the medium (either internal or external to the matrix) and physical and chemical properties of the therapeutic agent in the nanoparticle. In particular embodiments, release of a therapeutic agent encapsulated in a transferrin-conjugated nanoparticle of the instant invention is sustained for at least 2 months given the improved retention of the nanoparticle.

The biodegradable nanoparticle composition of the instant invention is composed of a therapeutic agent encapsulated in a nanoparticle, which is formulated using at least one biodegradable polymer. The nanoparticle further has attached to its surface, via a functional group exposed on the surface of the nanoparticle, a transferrin ligand. Nanoparticles are said to be biodegradable if the polymer of the nanoparticle dissolves or degrades within a period that is acceptable in the desired application (usually in vivo therapy), usually less than five years, and desirably less than one year, upon exposure to a physiological solution of pH 6-8 having a temperature of between 25° C. and 37° C. As such, a nanoparticle of the present invention can be composed of homopolymers, or copolymers prepared from monomers of polymers, wherein the copolymer can be of diblock, triblock, or multiblock structure. Suitable biodegradable polymers for formulating a nanoparticle of the instant invention include, but are not limited to, poly(lactide-co-glycolides), poly(lactic acid), poly(alkylene glycol), polybutylcyanoacrylate, poly(methylmethacrylate-co-methacrylic acid), poly-allylamine, polyanhydride, polyhydroxybutyric acid, or polyorthoesters and the like. In one embodiment, a biodegradable nanoparticle is formulated from at least one biodegradable polymer. In another embodiment, a biodegradable nanoparticle is formulated from at least two biodegradable polymers. In a further embodiment, a biodegradable nanoparticle is formulated from at least one biodegradable copolymer. In particular embodiments, the nanoparticle of the invention is composed of a copolymer of a poly(lactic acid) and a poly(lactide-co-glycolide). Particular combinations and ratios of polymers are well-known to the skilled artisan and any suitable combination can be used in the nanoparticle formulations of the present invention and may be dependent upon the therapeutic agent employed. In general, the resulting nanoparticle typically ranges in size from between 1 nm and 1000 nm, or more desirably between 50 nm and 150 nm as determined by, e.g. transmission electron microscopy (TEM).

To conjugate or operably attach the transferrin ligand to the surface of a nanoparticle of the present invention, a functional group is provided on the surface of the nanoparticle. A functional group, as used in the context of the instant invention is intended to include, but is not limited to, an amine group, a hydroxyl group, a carboxyl group, an aldehyde group, or an amide group. The functional group can be provided by any polymer of the nanoparticle including the biodegradable polymer or any other biocompatible polymer suitable for nanoparticle formulation, including but not limited to, poly(vinyl alcohol)(PVA), a polyethylene glycol such as d-α-tocopheryl polyethylene glycol 1000 succinate (Mu and Feng (2003) Pharma. Res. 20: 1864-1872) or human serum albumin (HAS) (Zambaux, et al. (1998) J. Control. Release 50:31-40). A fraction of emulsifier (e.g., PVA), used in the formulation of nanoparticles, has been shown to remain associated with nanoparticles despite repeated washing (surface-associated emulsifier) and forms the nanoparticle interface (Sahoo, et al. (2002) J. Control. Release 82:105-114). Accordingly, in one embodiment, the functional group is provided by an emulsifier. In particular embodiments, the functional group is provided on the surface of the nanoparticle by the emulsifier poly(vinyl alcohol).

A transferrin ligand can be attached to the functional group of the nanoparticle of the present invention using any standard conjugation method. Suitable conjugation methods include epoxy activation (Labhasetwar, et al. (1998) J. Pharm. Sci. 87:1229-34) or the use of 1-ethyl-3(3-dimethylaminopropyl) carbodiimide hydrochloride (EDC)/N-hydroxysulfo-succinimide (sulfo-NHS)(Hermanson (1996) Bioconjugate Techniques, Academic Press, San Diego, Calif., pp. 593-602). In a particular embodiment, epoxy activation is employed to conjugate transferrin to a functional group via an epoxy chain linker. In general, the nanoparticle surface is contacted with an epoxy compound which reacts with a functional group (e.g., a hydroxyl group of poly(vinyl alcohol)) associated with the nanoparticle surface. Suitable epoxy compounds include aromatic and aliphatic epoxides having two or more epoxy groups such as those based on glycidyl ethers, esters, acrylic polymers, urethane, epoxidized oils. In certain embodiments, the epoxy compound has at least two, at least three, at least four, or at least five epoxy groups. In particular embodiments, the epoxy compound has at least five epoxy groups to facilitate the linkage of high numbers of transferrin ligands to the surface of the nanoparticle. An exemplary epoxy compound is the sorbitol polyglycidyl ether known as DENACOL®. Epoxy activation of the nanoparticle creates multiple sites for reaction with a transferrin ligand and the epoxy chain acts as a linker between the nanoparticle surface and the transferrin ligand to avoid steric hindrance for interaction of the ligand with the cell membrane (Labhasetwar, et al. (1998) J. Pharm. Sci. 87:1229-34). Epoxy activation is particularly suitable as epoxy groups can react with many functional groups including amine, hydroxyl, carboxyl, aldehyde, and amide under suitable pH and buffer conditions.

As used herein, a transferrin ligand is any transferrin molecule which can be conjugated to a nanoparticle of the present invention to facilitate, enhance, or increase the transport of the nanoparticle into target cells as compared to a nanoparticle lacking transferrin on the surface. The term transferrin encompasses transferrin and isolated peptide fragments or biologically active portions thereof, analogs of transferrin and any biologically active portion thereof and any molecules and portions of molecules which bind a transferrin receptor. Transferrin can be commercially obtained as holo-transferrin or apo-transferrin, or can be recombinantly-produced or chemically synthesized using any method established in the art. In certain embodiments, a nanoparticle of the present invention has at least 100 transferrin molecules attached to the surface of a nanoparticle. In other embodiments, a nanoparticle of the present invention has from about 100 to 1000 transferrin molecules per nanoparticle or from about 300 to 700 transferrin molecules per nanoparticle. In one embodiment, a nanoparticle of the present invention has about 500 transferrin molecules per nanoparticle.

A transferrin ligand on the surface of a nanoparticle of the invention can be unlabeled or labeled, e.g., with a radionuclide. Advantageously, the nanoparticles of the instant invention can be used to deliver a radionuclide to a target cell in combination with the encapsulated therapeutic agent. A nanoparticle formulation combining a radionuclide and an encapsulated therapeutic agent would provide effective therapy with reduced toxicity in the treatment of cancer. Beta-emitters such as ⁵⁹Fe are particularly suitable radionuclides for use in the context of the instant invention as their range of beta-emissions extends for several millimeters from the source. Moreover, therapeutic radioisotopes such as ¹³¹I for direct labeling of transferrin, and ⁹⁰Y, ¹⁷⁷Lu, ¹⁸⁶Re or ¹⁸⁸Re, as surrogates for ⁵⁹Fe-holo-transferrin, can also be used to create a crossfire effect, destroying tumor cells to which the nanoparticles are not directly bound or when the diffusion of the nanoparticles into the tumor is limited. Beta-particle nanoparticle therapy is well-suited for bulky tumors or large-volume disease.

A nanoparticle formulation of the present invention can further contain other components such as plasticizers to facilitate sustained-release of the encapsulated active agent by maintaining the structure of the nanoparticle. Release of molecules (e.g., proteins, DNA or oligonucleotides) from nanoparticles formulated from block copolymers is, in general, not continuous. Typically, there is an initial release followed by a very slow and insignificant release thereafter. Not to be bound by theory, it is contemplated that the release profile may be as a result of the rapid initial drop in the molecular weight of the polymer which reduces the glass transition temperature of the polymer to below body temperature (37° C.); the glass transition temperature of copolymers prior to release is above body temperature (˜45 to 47° C.). Moreover, with degradation, these polymers become softer thereby closing the pores which are created during the initial release phase (due to the release of active agent from the surface). Therefore, an inert plasticizer can be added to a nanoparticle formulation disclosed herein to maintain the glass transition temperature above 37° C. despite a decline in molecular weight of the polymer with time. In this manner, the pores remain open and facilitate a continuous release of the encapsulated therapeutic agent. Suitable plasticizers are generally inert and can be food/medical grade or non-toxic plasticizers including, but not limited to, triethyl citrate (e.g., CITROFLEX®, Morflex Inc., Greensboro, N.C.), glyceryl triacetate (e.g., Triacetin, Eastman Chemical Company, Kingsport, Tenn.), L-tartaric acid dimethyl ester (i.e., dimethyl tartrate, DMT) and the like. A particularly suitable plasticizer is L-tartaric acid dimethyl ester.

The amount of plasticizer employed in a nanoparticle composition can range from about 5 to 40 weight percent of the nanoparticle, more desirably from about 10 to 20 weight percent of the nanoparticle.

For certain therapeutic agents, whose targets are cytoplasmic, nuclear or mitochondrial, it is necessary that the drug carrier system employed escapes the endosomal compartment to achieve an optimal pharmacologic effect (Panyam and Labhasetwar (2004) Current Drug Delivery 1:235-247). For example, paclitaxel, a potent antineoplastic agent, which exerts its pharmacological action by binding to microtubules (Jordan and Wilson (2004) Nat. Rev. Cancer 4:253-265) must be in the cytoplasmic compartment to be effective. Doxorubicin is another antineoplastic agent which acts by intercalation with nuclear DNA, and therefore, must escape the endosomal vesicles to the cytosol for its subsequent diffusion into the nucleus. Apart from the effect of the membrane-associated efflux transporter proteins (e.g., P-gp), drug resistance in cancer cells has been attributed to sequestering of antineoplastic agents into these acidic endosomal vesicles, thus reducing their levels in the cytosolic compartment (Brigger, et al. (2002) supra). In addition to drug disposition to the right intracellular target compartment, the duration of drug retention at the target site may be a critical factor in certain disease conditions to achieve the desired therapeutic outcome. For example, sustained-inhibition of vascular smooth muscle proliferation is necessary to prevent hyperplasia in the injured artery following balloon angioplasty or stenting (Kavanagh, et al. (2004) Pharmacol. Ther. 102:1-15). Similarly, cancer is a disease condition that requires chronic drug therapy to completely regress the tumor growth and to avoid its relapse (Jang, et al. (2003) Pharm. Res. 20:1337-1350). Therefore, in addition to the targeting of antineoplastic agents, their retention at therapeutic doses in tumor cells is equally important.

Accordingly, transferrin-conjugated nanoparticle formulations that result in greater cytoplasmic localization and retention of therapeutic agents will enhance the efficacy of a variety of therapeutic agents in a variety of organs, tissues, or cells. Thus, the present invention further relates to a method for increasing the efficacy of a therapeutic agent by encapsulating the therapeutic agent in a transferrin-conjugated nanoparticle and administering the therapeutic agent to a subject in need of treatment with the therapeutic agent. As used herein, an increase in efficacy is defined as a 30%, 40%, 50%, 60%, 70%, 80%, 90% or more increase in the intended effect, i.e., intracellular uptake and activity of a specified amount of therapeutic agent. The use of a transferrin-conjugated nanoparticle containing a specified amount of a therapeutic agent results in higher intracellular levels and greater retention of the therapeutic agent when compared to the same specified amount of therapeutic agent encapsulated in unconjugated nanoparticles or free in solution. As such, the therapeutic agent encapsulated in a transferrin-conjugated nanoparticle can be administered in lower doses or less frequent dosing schedules than less efficacious formulations thereby reducing side effects, toxicity, and costs associated with administering higher dosages of the therapeutic agent.

The transferrin receptor is expressed in nucleated cells in the body, such as red blood cells, erythroid cells, hepatocytes, intestinal cells, monocytes (macrophages), brain, the blood-brain barrier (also blood-testis and blood-placenta barriers), and also in some insects and certain bacteria (Levay and Viljoen (1995) Haematologica 80:252-267; Lönnerdal and Iyer (1995) Annu. Rev. Nutr. 15:93-110; Schryvers, et al. (1998) Adv. Exp. Med. Biol. 433:123-133; Qian, et al. (1998) Neurosci. Lett. 251:9-12; Qian, et al. (1999) Exp. Brain Res. 129:473-476; Qian, et al. (2000) Cell Mol. Biol. 46:541-548; Moos and Morgan (2000) Cell Mol. Neurobiol. 20:77-95). Transferrin fused to mouse-human chimeric IgG3 crosses the blood-brain barrier to target the brain (Shin, et al. (1995) Proc. Natl. Acad. Sci. USA 92:2820-2824). Therefore, in particular embodiments, a transferrin-conjugated nanoparticle of the instant invention will be particularly useful for delivering therapeutic agents to the brain for the prevention or treatment of neurodegenerative diseases and brain injury. Further, the transferrin receptor is expressed on rapidly dividing cells, with 10,000 to 100,000 molecules per cell commonly found on tumor cells or cell lines in culture (Inoue, et al. (1993) J. Cell Physiol. 156:212-217). Thus, the use of transferrin is especially advantageous in connection with delivery of cancer therapeutics as efficient, targeted delivery of cancer therapeutics to cancer cells can be achieved with reduced side effects.

A therapeutic agent, in the context of the instant invention, encompasses any natural or synthetic, organic or inorganic molecule or mixture thereof for preventing or treating a disease or condition in a subject. As used herein, a therapeutic agent includes any compound or mixture of compounds which produces a beneficial or useful result. Therapeutic agents are distinguishable from such components as vehicles, carriers, diluents, lubricants, binders and other formulating aids, and encapsulating, delivery or otherwise protective components. Examples of therapeutic agents include locally or systemically acting therapeutic agents which can be administered to a subject in need of treatment (i.e., exhibiting signs or symptoms associated with a particular disease or condition) according to standard methods of delivering nanoparticles (e.g., oral, topical, intralesional, injection, such as subcutaneous, intradermal, intratumoral, intramuscular, intraocular, or intra-articular injection, and the like). Examples of therapeutic agents for the prevention or treatment of diseases and conditions include, but are not limited to, anti-oxidants (e.g., superoxide dismutase, catalase, glutathione peroxidase, glutathione reductase, glutathione-S-transferase), anti-infectives (including antibiotics, antivirals, fungicides, scabicides or pediculicides), antiseptics (e.g., benzalkonium chloride, benzethonium chloride, chlorohexidine gluconate, mafenide acetate, methylbenzethonium chloride, nitrofurazone, nitromersol and the like), steroids (e.g., estrogens, progestins, androgens, adrenocorticoids, and the like), therapeutic polypeptides (e.g. insulin, erythropoietin, morphogenic proteins such as bone morphogenic protein, and the like), analgesics and anti-inflammatory agents (e.g., aspirin, ibuprofen, naproxen, ketorolac, COX-1 inhibitors, COX-2 inhibitors, and the like), cancer therapeutic agents (e.g., paclitaxel, mechliorethamine, cyclophosphamide, fluorouracil, thioguanine, carmustine, lomustine, melphalan, chlorambucil, streptozocin, methotrexate, vincristine, bleomycin, vinblastine, vindesine, dactinomycin, daunorubicin, doxorubicin, tamoxifen, and the like), narcotics (e.g., morphine, meperidine, codeine, and the like), local anesthetics (e.g., the amide- or anilide-type local anesthetics such as bupivacaine, dibucaine, mepivacaine, procaine, lidocaine, tetracaine, and the like), antiangiogenic agents (e.g., combrestatin, contortrostatin, anti-VEGF, and the like), neuroprotective agents (e.g., neurotrophins such as BDNF), polysaccharides, vaccines, antigens, nucleic acids (e.g., DNA and other polynucleotides, antisense oligonucleotides, and the like), etc. As exemplified herein, the therapeutic agent can be encapsulated within the nanoparticle during formulation of the nanoparticle, or alternatively, can be added after the formulation of the nanoparticle. Any suitable method for incorporating the therapeutic agent can be employed which maximizes the loading of the therapeutic agent in the nanoparticle. Nanoparticles with different drug release rates can also be formulated to increase the effect of the encapsulated drug.

As will be appreciated by the skilled artisan, the nanoparticle compositions of the present invention can further contain additional fillers, excipients, binders, etc. depending on, e.g., the route of administration and the therapeutic agents used. A generally recognized compendium of such ingredients and methods for employing the same is Remington: The Science and Practice of Pharmacy, Alfonso R. Gennaro, editor, 20th ed. Lippingcott Williams & Wilkins: Philadelphia, Pa., 2000.

To illustrate the utility of the compositions and methods of the present invention, paclitaxel-encapsulated, transferrin-conjugated nanoparticles were formulated and analyzed for uptake and in vitro and in vivo antiproliferative activity. A suspension of nanoparticles (100 μg/mL) was incubated with MCF-7 or PC3 cells for 1 hour, cells were washed, and nanoparticle levels in cells were determined by HPLC. To determine the competitive inhibition of uptake of transferrin-conjugated nanoparticles, an excess dose of free transferrin (50 μg) was added in the medium prior to incubation with transferrin-conjugated nanoparticles. Cellular uptake of transferrin-conjugated nanoparticles was about two- to three-fold greater than unconjugated nanoparticles. The specificity of transferrin-mediated binding of conjugated nanoparticles was evident from the reduced uptake of transferrin-conjugated nanoparticles in the presence of free transferrin (Table 1).

TABLE 1

| Treatment | Uptake (μg/mg Cell Protein) |
|---|---|
| Unconjugated nanoparticle | 13 ± 2 |
| Transferrin-conjugated | 35 ± 5** |
| nanoparticle | |
| Transferrin-conjugated | 15 ± 2* |
| nanoparticle plus free | |
| transferrin | |

Data as mean ± s.e.m. (n = 6).
*p < 0.05 transferrin-conjugated nanoparticles plus free transferrin versus nanoparticles,
**p < 0.005 transferrin-conjugated nanoparticles versus nanoparticles.

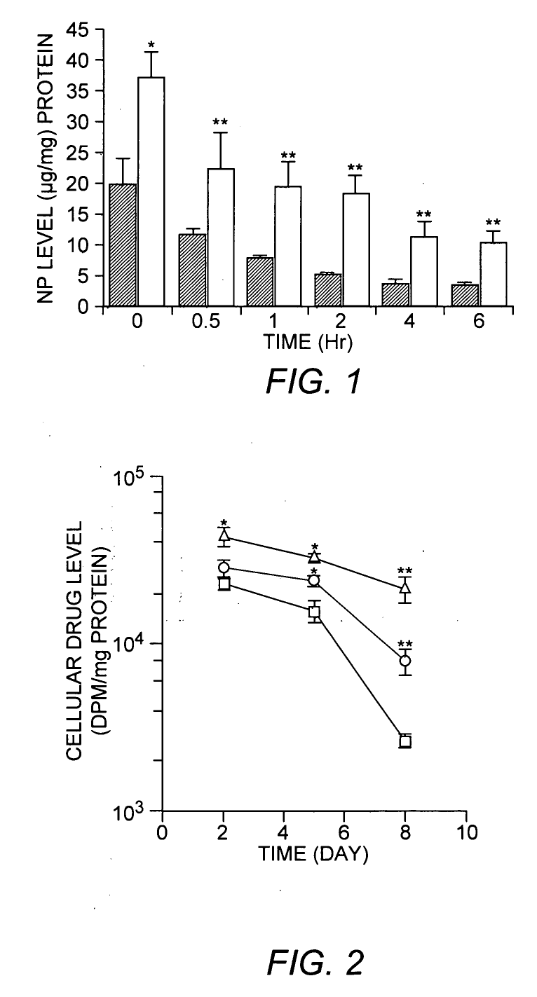

Exocytosis analysis demonstrated reduced exocytosis of transferrin-conjugated nanoparticles compared to that of unconjugated nanoparticles. More than 75% of the internalized unconjugated nanoparticles underwent exocytosis during 2 hours as compared to 50% of transferrin-conjugated nanoparticles (FIG. 1). Thus, the higher uptake and reduced exocytosis resulted in overall greater cellular retention of transferrin-conjugated nanoparticles as compared to unconjugated nanoparticles.

Confocal laser scanning microscopy of MCF-7 cells demonstrated internalization of paclitaxel within 4 hours of incubation; however, the fluorescence intensity was reduced slowly with incubation time in cells treated with paclitaxel in solution. On the other hand, the fluorescence intensity increased with incubation time in cells treated unconjugated paclitaxel-encapsulated nanoparticles or paclitaxel-encapsulated transferrin-conjugated nanoparticles. Intracellular drug retention was observed for a longer duration in case of paclitaxel-encapsulated transferrin-conjugated nanoparticles than in unconjugated paclitaxel-encapsulated nanoparticle-treated cells, whereas cells treated with paclitaxel in solution demonstrated insignificant fluorescent activity at 5 days. The increase in fluorescence intensity with incubation time in cells treated with nanoparticles was due to the slow release of the encapsulated drug from the nanoparticles which were localized inside the cells. The difference in the fluorescence intensity between cells treated with unconjugated paclitaxel-encapsulated nanoparticles and paclitaxel-encapsulated, transferrin-conjugated nanoparticles was clear at 8 days following treatment. Cells treated with transferrin-conjugated nanoparticles at 8 days demonstrated higher fluorescence intensity than cells treated with unconjugated nanoparticles. The difference in drug levels between different treatment groups was apparent when their levels were quantified using tritium-labeled paclitaxel. The drug level was more sustained in cells treated with paclitaxel-encapsulated transferrin-conjugated nanoparticles than in cells treated with drug in solution or unconjugated nanoparticles (FIG. 2), thus substantiating confocal microscopic observations.

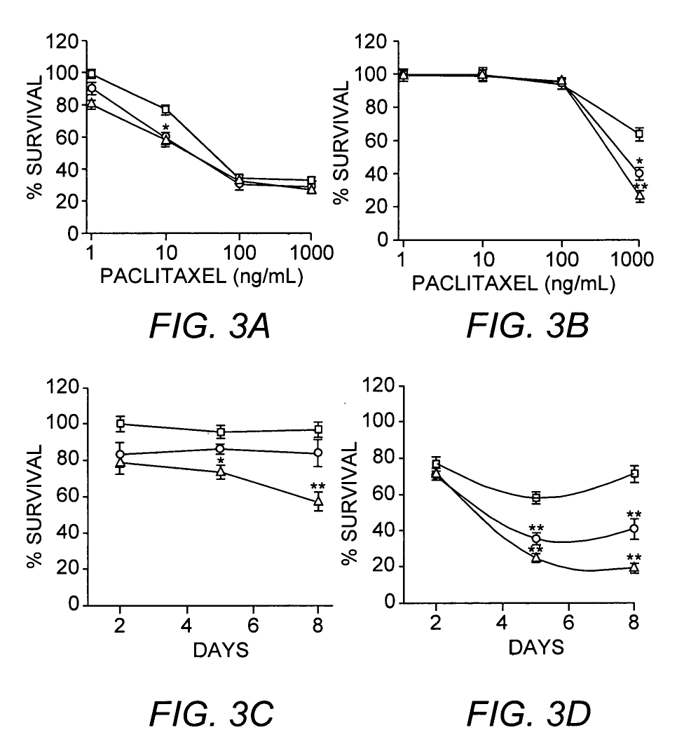

In a dose-response study using MCF-7 cells and the lowest dose of paclitaxel studied (1 ng/mL), paclitaxel-encapsulated transferrin-conjugated nanoparticles demonstrated greater antiproliferative activity than paclitaxel in solution or unconjugated paclitaxel-encapsulated nanoparticles (FIG. 3A). At higher doses of drug, all the treatment groups demonstrated almost similar inhibitory effect. Although unconjugated paclitaxel-encapsulated nanoparticles demonstrated greater antiproliferative activity than paclitaxel in solution, there was no significant change in the antiproliferative effect of the drug with incubation time in these groups. In contrast, paclitaxel-encapsulated, transferrin-conjugated nanoparticles demonstrated an increase in the antiproliferative with incubation time and the effect was more significant at 8 days following the treatment (FIG. 3B). The antiproliferative effect of the drug in nanoparticles can be considered greater than that with drug in solution because only a fraction of the encapsulated drug is released from nanoparticles during the experimental time period (˜23% in 5 days or 30% in 8 days, based on in vitro release analysis).

MCF-7/Adr cells did not exhibit a decrease in proliferation up to 100 ng/mL drug concentration in all the treatment groups; however, at a higher dose (1000 ng/mL), paclitaxel-encapsulated, transferrin-conjugated nanoparticles demonstrated relatively greater antiproliferative effect than paclitaxel in solution or unconjugated paclitaxel-encapsulated nanoparticles (˜70% vs. 20% for paclitaxel in solution and paclitaxel-encapsulated, transferrin-conjugated nanoparticles; and 55% for unconjugated paclitaxel-encapsulated nanoparticles) (FIG. 3C). Cells treated with paclitaxel-encapsulated, transferrin-conjugated nanoparticles demonstrated an increase in inhibition of cell proliferation with incubation time, whereas cells treated with paclitaxel in solution demonstrated transient inhibition in cell growth up to 5 days following treatment but regained the growth thereafter. In the case of unconjugated paclitaxel-encapsulated nanoparticles, the antiproliferative effect was more sustained and greater than that with paclitaxel in solution but it was lower than paclitaxel-encapsulated, transferrin-conjugated nanoparticles (FIG. 3D). Further, the incubation time had greater antiproliferative effect with paclitaxel-encapsulated, transferrin-conjugated nanoparticles in MCF-7/Adr cells than in MCF-7 cells (FIG. 3B vs. FIG. 3D). Although the dose of paclitaxel used in the resistant cell line was 100-fold greater than that used in the sensitive cell line, the observation indicates that the effect of drug retention on antiproliferative activity has a greater effect in resistant cells than in non-resistant cells.

In similar studies using PC3 prostate cancer cells, the lowest dose (1 ng/mL) of the drug used demonstrated ˜70% inhibition in cell proliferation with paclitaxel-encapsulated, transferrin-conjugated nanoparticles as compared to 25% inhibition with the same dose of paclitaxel in solution and 35% inhibition in unconjugated paclitaxel-encapsulated nanoparticles. A 10-fold higher dose of the drug in solution (10 ng/mL) was required to achieve similar antiproliferative activity as that with paclitaxel-encapsulated, transferrin-conjugated nanoparticles. Curve fitting was used to calculate the IC₅₀ (concentration required for 50% inhibition of growth) of the drug from the dose response study. The IC₅₀ of the drug was about 5-fold lower with paclitaxel-encapsulated, transferrin-conjugated nanoparticles than that with unconjugated paclitaxel-encapsulated nanoparticles or paclitaxel in solution (IC₅₀=6×10⁻⁴ μM for paclitaxel-encapsulated, transferrin-conjugated nanoparticles vs. 3.1×10⁻³ μM for unconjugated paclitaxel-encapsulated nanoparticles and 3.5×10⁻³ μM for paclitaxel in solution). Since the medium control and control nanoparticles demonstrated similar growth curves, the antiproliferative effect seen with the drug-loaded nanoparticles in PC3 cells was due to the activity of the encapsulated drug.

A single-dose intratumor injection of paclitaxel-encapsulated, transferrin-conjugated nanoparticles either at 24 mg/kg and 12 mg/kg or unconjugated paclitaxel-encapsulated nanoparticles at 24 mg/kg dose, induced significant tumor inhibition as compared to that with a paclitaxel CREMOPHOR® EL formulation at 24 mg/kg dose. The most significant result was the complete regression of tumor growth with paclitaxel-encapsulated, transferrin-conjugated nanoparticles at 24 mg/kg drug dose. In addition, the number of animals survived in the paclitaxel-encapsulated, transferrin-conjugated nanoparticle treatment group was significantly greater than that in the unconjugated paclitaxel-encapsulated nanoparticle (24 mg/kg) or paclitaxel CREMOPHOR® EL formulation groups.

Having demonstrated the efficacy of using an antineoplastic agent encapsulated in a transferrin-conjugated nanoparticle, the present invention also encompasses methods for decreasing tumor cell proliferation and treating cancer by administering to a subject having or at risk of having cancer an effective amount of a cancer therapeutic encapsulated in a transferrin-conjugated, biodegradable nanoparticle.

Treatment typically involves the steps of first identifying a subject having or at risk of having a cancer. Individuals having cancer generally refers to subjects who have been diagnosed with a cancer and require treatment, whereas individuals at risk of having a cancer may have a family history of cancer or exhibit one or more signs or symptoms associated with such a cancer and require prevention of the same. Once such a subject is identified using, for example, standard clinical practices, the subject is administered a transferrin-conjugated nanoparticle composition containing an effective amount of cancer therapeutic, e.g., as disclosed supra, which results in a decrease in the signs or symptoms or duration of the cancer being treated. In most cases, the subject being treated will be a human being, but treatment of agricultural animals, e.g., livestock and poultry, and companion animals, e.g., dogs, cats and horses, is expressly covered herein. Cancers which can be treated in accordance with the method of the invention include, but are not limited, to cancers of the lung, colon, breast, prostate, brain, head, neck, ovary, uterus, and pancreas.

The selection of the dosage or effective amount of the agent is that which has the desired outcome of reducing or reversing at least one sign or symptom of cancer or increasing survival. For example, depending on the cancer, some of the general signs or symptoms can include a tumor, increased pain perception, weakness, abdominal pain, and anemia.

The cancer therapeutic agent-encapsulated, transferrin-conjugated nanoparticle composition of the present invention can be delivered via various routes and to various sites in a mammalian, particularly human, body to achieve a particular effect. One skilled in the art will recognize that, although more than one route can be used for administration, a particular route can provide a more immediate and more effective reaction than another route.

Those of ordinary skill in the art can readily optimize effective doses and administration regimens as determined by good medical practice and the clinical condition of the individual patient. Regardless of the manner of administration, it can be appreciated that the actual preferred amounts of therapeutic agent in a specific case can vary according to the particular therapeutic agent and the route of administration. The specific dose for a particular patient depends on age, body weight, general state of health, on diet, on the timing and route of administration, on the rate of excretion, and on medicaments used in combination and the severity of the particular disorder to which the therapy is applied. Dosages for a given subject can be determined using conventional considerations, e.g., by customary comparison of the differential activities of the selected therapeutic agent encapsulated in a transferrin-conjugated nanoparticle and of the same agent in solution, such as by means of an appropriate conventional pharmacological protocol.

Based upon the improved retention and increased therapeutic efficacy provided by a nanoparticle of the instant invention, it is contemplated that other ligands to other receptors may also be useful for targeting nanoparticles to cells.

The invention is described in greater detail by the following non-limiting examples.

### Example 1

### Materials

Poly (D,L-lactide-co-glycolide) (PLGA, molecular weight 23,000 Dalton, copolymer ratio 50:50) was purchased from Birmingham Polymers, Inc. (Birmingham, Ala.). CREMOPHOR® EL, zinc tetrafluoroborate hydrate, holo-transferrin, and polyvinyl alcohol (PVA, average molecular weight 30,000-70,000 Da), TWEEN™ 20 were purchased from Sigma Chemical Co. (St. Louis, Mo.). Paclitaxel was purchased from Hauser Chemical Company (Boulder, Colo.), whereas tritium-labeled paclitaxel was purchased from Moravek Biochemicals (Brea, Calif.). TEXAS RED™-conjugated transferrin and OREGON GREEN™ 488-labeled paclitaxel were purchased from MOLECULAR PROBES™ (Eugene, Oreg.). DENACOL® EX-521 (molecular weight 742, Pentaepoxy) was from Nagase Chemicals Ltd. (Tokyo, Japan). 6-Coumarin was purchased from Polyscience Inc. (Warrington, Pa.). All salts used in the preparation of buffers were from Fisher Scientific (Pittsburgh, Pa.). All aqueous solutions were prepared with distilled and deionized water (Water Pro Plus; Labconco, Kansas City, Mo.).

### Example 2

### Formulation of Nanoparticles

Nanoparticles containing paclitaxel were formulated using an emulsion-solvent evaporation technique. In brief, a solution of 90 mg PLGA polymer and 6 mg paclitaxel in 3 mL of chloroform was emulsified into 12 mL of 5% weight/volume aqueous solution of PVA to form an oil-in-water emulsion. Emulsification was carried out using a micro-tip probe sonicator set at 55 Watts of energy output (XL 2015 SONICATOR® ultrasonic processor; Misonix Inc., Farmingdale, N.Y.) for 2 minutes over an ice bath. The emulsion was subsequently stirred for ˜18 hours at room temperature on a magnetic stir plate to allow the evaporation of chloroform. Nanoparticles thus formed were recovered by ultracentrifugation at 30,000×g or 110,000×g for 20 minutes at 4° C. (OPTIMA™ LE-80K; Beckman Instruments, Inc., Palo Alto, Calif.), washed twice with water to remove PVA and unencapsulated drug, and then lyophilized for 2 days (VIRTIS® Freeze Dryer; VIRTIS® Company, Inc., Gardiner, N.Y.). To determine cellular uptake, nanoparticles containing a fluorescent dye (6-coumarin) were employed. The dye solution (50 μg of 6-coumarin in 50 μL chloroform) was added to the polymer solution prior to emulsification. The incorporated dye acts as a probe for nanoparticles, and offers a sensitive method to quantitatively determine intracellular nanoparticle uptake (Panyam, et al. (2003) Int. J. Pharm. 262:1-11). To determine intracellular retention of drug, nanoparticles containing tritium- or fluorescent-labeled paclitaxel were prepared using the same procedure as above.

### Example 3

### Transferrin-Conjugated Nanoparticles

Transferrin was conjugated to nanoparticies using two steps; nanoparticles were activated by epoxy compound (Labhasetwar, et al. (1998) J. Pharm. Sci. 87:1229-34) and activated nanoparticles were conjugated to transferrin.

Surface Activation of Nanoparticles. Briefly, 20 mg of nanoparticles (encapsulated with either 6-coumarin or paclitaxel) was suspended in 4 mL borate buffer (50 mM, pH 5.0) by sonication using a micro-tip probe sonicator as above for 30 seconds over an ice bath. Six milligrams of zinc tetrafluoroborate hydrate (catalyst) was added to the nanoparticle suspension followed by a solution of DENACOL® (10 mg in 2 mL borate buffer). Stirring of the nanoparticle suspension was continued for 30 minutes at 37° C., nanoparticles were separated by ultracentrifugation at 30,000×g or 110,000×g for 20 minutes at 4° C., and then washed three times with borate buffer to remove unreacted DENACOL®.

Transferrin Conjugation to Epoxy Activated Nanoparticles. Epoxy-activated nanoparticles (20 mg) were suspended in 5 mL of borate buffer (50 mM, pH 5.0) as above. A solution of transferrin in borate buffer (10-15 mg/mL) was added to the suspension of nanoparticles, and the reaction was carried out at 37° C. for 2 hours with low speed stirring on a magnetic stir plate. Excess transferrin was removed, first by ultracentrifugation at 110,000×g for 20 minutes at 4° C. as above followed by overnight dialysis (SPECTROPORE®, molecular weight cut off 100 KDa) The suspension of nanoparticles from the bag was collected, frozen at −70° C., and then lyophilized for 48 hours.

The average number of transferrin molecules conjugated to nanoparticles was calculated indirectly by measuring the amount of transferrin that was not conjugated to nanoparticles. For this purpose TEXAS RED™-conjugated transferrin was used and the washings were collected to determine the amount of transferrin that did not bind to nanoparticles. The average number of transferrin molecules conjugated per nanoparticle was calculated by dividing the number of transferrin molecules bound to nanoparticles by the calculated average number (n) of nanoparticles using the following equation (Olivier, et al. (2002) Pharm. Res. 233:51-9): n=6 m/(π×D³×ρ), wherein m is the nanoparticle weight, D is the number based on mean nanoparticle diameter determined by photon correlation spectroscopy, and ρ=nanoparticle weight per volume unit (density), estimated to be 1.1 g/cm³ based on the polymer density.

### Example 4

### Physical Characterization of Nanoparticles

The ¹H-NMR spectrum of transferrin-conjugated nanoparticles was recorded on a Bruker AMX 500 spectrophotometer in D₂O. Particle size and size distribution was determined by photon correlation spectroscopy using quasi-elastic laser light scattering equipment. A dilute suspension of nanoparticles (100 μg/mL) was prepared in double-distilled water and sonicated as above on an ice bath for 30 seconds. The sample was subjected to particle size analysis in a ZETAPLUS™ particle size analyzer (Brookhaven Instrument Corp., Holtsville, N.Y.). Nanoparticles were also evaluated for size by transmission electron microscope (Philips/FEI, Briarcliff Manor, N.Y.). A sample of nanoparticles (0.5 mg/mL) was suspended in water and particles were visualized after negative staining with 2% weight/volume uranyl acetate (Electron Microscopy Services, Ft. Washington, Pa.). To measure zeta potential, a suspension of nanoparticles was prepared in 0.001 M HEPES buffer (pH 7.4) as above and zeta potential was measured immediately using a ZETAPLUS™ zeta potential analyzer. The amount of PVA associated with nanoparticles was determined using a well-established colorimetric method (Sahoo, et al. (2002) supra). Paclitaxel loading in nanoparticles was determined by extracting the drug from nanoparticles by shaking a sample containing the same (5 mg) with 2 mL of methanol at 37° C. for 48 hours at 150 rpm using an ENVIRON® orbital shaker (Lab Line, Melrose Park, Ill.). Nanoparticles were centrifuged at 14,000 rpm for 10 minutes in a microcentrifuge, and 100 μL of the supernatant was diluted to 500 μL with methanol and analyzed for paclitaxel levels using HPLC. The drug release from nanoparticles was carried using double-diffusion chambers in phosphate-buffered saline (PBS; 154 mM, pH 7.4) containing 0.1% TWEEN™ 20 to maintain sink conditions.

Drug levels were quantitated using HPLC. The HPLC system (Shimadzu Scientific Instrument, Inc., Columbia, Md.) consisted of a Curosil-B column (250×3.2 mm²) with 5 μm packing (Phenomenex, Torrance, Calif.). The mobile phase consisted of a mixture of ammonium acetate (10 mM, pH 4.0) and acetonitrile in the ration of 55:45 (volume:volume) and was delivered at a flow rate of 0.4 mL/minute with a pump (Model LC-10AT). A 20 μL sample was injected using an autoinjector (Model SIL-10A) and paclitaxel levels were quantified by UV detection (λ=228 nm, Model SPD-10A VP). A standard plot for paclitaxel (0-20 μg/mL) was prepared under identical conditions.

The physical state of paclitaxel encapsulated in nanoparticles was characterized with differential scanning calorimetric thermogram analysis (Shimadzu, DSC-50 differential calorimeter fitted with a Shimadzu TA-50 data processor, Columbia, Md.). Each sample (8 mg; paclitaxel, placebo nanoparticles, and paclitaxel-loaded nanoparticles) was sealed in standard aluminum pans with lids and purged with pure dry nitrogen at a flow rate of 20 mL/minute. A temperature ramp speed was set at 10° C./minute and the heat flow was recorded from 0 to 350° C.

Transferrin was chemically coupled to the hydroxyl groups of the PVA associated with nanoparticle surface (PVA associated with nanoparticles=5±1.2% weight/weight) through a multifunctional epoxy compound DENACOL® EX 521. It has five epoxy groups, at least one of its epoxy groups was conjugated to hydroxyl group of PVA associated with nanoparticles and the other epoxy groups to the amine group of transferrin. The chemical composition of transferrin-conjugated nanoparticles by ¹H-NMR spectroscopy demonstrated a peak at 2.2 ppm that confirmed that an amino group of transferrin was conjugated to epoxy groups. The amount of conjugated transferrin was determined to be 2.9% weight/weight of the nanoparticle mass, which represents approximately 440 transferrin molecules per nanoparticle.

Nanoparticles demonstrated a mean hydrodynamic diameter of 216-220 nm with polydispersity index of ˜0.1 to 0.12, indicating uniform particle size distribution. Conjugation with transferrin slightly increased the hydrodynamic mean diameter of particles (˜6 nm) and their zeta potential was slightly more negative than unconjugated nanoparticles (−8.12±2.8 mV vs. −9.34±2.6 mV). The mean particle size of these nanoparticles with TEM was approximately 110 nm±6 (mean±SD, particles counted from eight different TEM fields). The difference in particle size measured by laser light scattering and by TEM has been established in the art (Prabha, et al. (2002) Int. J. Pharm. 244:105-115). The laser light scattering measures hydrodynamic diameter, and hydration of the surface-associated PVA probably contributes towards the hydrodynamic diameter of nanoparticles. Paclitaxel loading in nanoparticles was 5.4% weight/weight with an encapsulation efficiency of 86% (i.e., 86% of the drug added in formulation was entrapped in nanoparticles). Nanoparticles demonstrated sustained-release of the encapsulated drug, with ˜30% cumulative drug release occurring in one week and about ˜60% in two months. Thus, the estimated drug available from this formulation, based on the drug loading and in vitro release rate is ˜0.5 ng/μg nanoparticle/day.

Selection of polymer PLGA of 50:50 (lactide or glycolide ratio) and molecular weight 23,000 for the formulation of nanoparticles was based on the solid-state solubility of paclitaxel in polymer, i.e., drug solubility in solid polymer without phase separation. It has been demonstrated that encapsulation efficiency of hydrophobic agents in nanoparticles depends on the solid-state solubility of the drug in polymer, and is higher for a polymer that demonstrates greater solid-state drug-polymer solubility (Panyam, et al. (2004) J. Pharm. Sci. 93:1804-14). The DSC thermograms demonstrated that only pure paclitaxel had an endothermic peak of melting at 215-217° C. but no peak was observed in the range 150-250° C. for the control or drug-loaded nanoparticles. The results thus indicate that paclitaxel formulated in nanoparticles was in an amorphous or disordered-crystalline phase of a molecular dispersion or a solid-state solution in the polymer matrix (Mu, et al. (2002) J. Control. Release 80:129-144).

### Example 5

### Cell Culture

PC3 prostate cancer cells and MCF-7 breast cancer cells were obtained from American Type Culture Collection (Manassas, Va.) and were grown in RPMI 1640 supplemented with 10% fetal bovine serum, 100 μg/mL penicillin G and 100 μg/mL streptomycin (GIBCO BRL, Grand Island, N.Y.) at 37° C. in a humidified, 5% CO₂ atmosphere. MCF-7/Adr cells were maintained on DMEM medium supplemented with 10% Cosmic calf serum (Hyclone, Logan, Utah) and 100 μg/mL penicillin G and 100 μg/mL streptomycin (GIBCO BRL, Grand Island, N.Y.) at 37° C. in a humidified, 5% CO₂ atmosphere.

### Example 6

### Intracellular Uptake of Nanoparticles

Plates (24-well) were seeded with PC3 or MCF-7 cells at 5×10⁴ per well density and cells were allowed to attach for 24 hours. The medium in each well was replaced with 1 mL of freshly prepared suspension of nanoparticles in medium (100 μg/well) and the plates were incubated for one hour. To examine the specificity of transferrin receptor-mediated nanoparticle uptake, cells were incubated for 1 hour with excess dose of free transferrin (50 μg) prior to incubation with transferrin-conjugated nanoparticles. Cells were then washed three times with phosphate-buffered saline (PBS) to remove nanoparticles which were not internalized, and then incubated with 0.1 mL of 1× cell culture lysis reagent (PROMEGA®, Madison, Wis.) for 30 minutes at 37° C. A 5 μL aliquot of each cell lysate was used for cell protein determination using a Bradford protein assay (BIO-RAD®, Hercules, Calif.) and the remaining portion was lyophilized. The dye from nanoparticles in the lyophilized samples was extracted by shaking each sample with 1 mL methanol at 37° C. for 48 hours at 150 rpm using an ENVIRON® orbital shaker (Lab Line, Melrose Park, Ill.). The samples were centrifuged at 14,000 rpm for 10 minutes in a microcentrifuge (EPPENDORF® 5417R; Brinkmann Instruments, Westbury, N.Y.) to remove cell debris. The supernatant was analyzed for 6-coumarin levels by HPLC according to standard methods (Sahoo, et al. (2002) supra). A standard plot with different concentrations of nanoparticles was constructed simultaneously under similar conditions to determine the amount of nanoparticles in cell lysate. The data was normalized to per mg cell protein.

### Example 7

### Exocytosis of Nanoparticles

Exocytosis was carried out according to standard protocols (Panyam and Labhasetwar (2003) supra). In brief, 24-well plates were seeded with MCF-7 cells at 50,000 per well density and cells were allowed to attach for 24 hours. The medium in each well was replaced with 1 mL of freshly prepared nanoparticle suspension in medium (100 μg/well) and the plates were incubated for 1 hour. A formulation of nanoparticles loaded with 6-coumarin dye was used. Cells were washed three times with PBS to remove nanoparticles which were not internalized. The intracellular nanoparticle levels after washing of cells were taken as the uptake at zero time point. The cells in other wells were incubated with fresh medium and, at different time points, the medium was removed, cells were washed three times with PBS and lysed with 0.1 mL of 1× cell culture lysis reagent (PROMEGA®, Madison, Wis.) to determine nanoparticle levels in cell lysates (Panyam and Labhasetwar (2003) supra).

### Example 8

### Mitogenic Assay

MCF-7 or MCF-7/Adr cells were seeded at 4,000 per well density in 96-well plates and allowed to attach for 24 hours. Similarly, PC3 cells were seeded at 5,000 per well in 96-well plates and allowed to attach for 24 hours. A stock solution of paclitaxel was prepared in ethanol (1 mg/mL) and stored at −70° C. Different aliquots of the above stock paclitaxel solution were added to the culture medium to achieve desired drug concentrations. The concentration of ethanol in the medium was kept <0.1% so that it had no effect on cell proliferation (Kelland and Abel (1992) Cancer Chemother. Pharmacol. 30:444-50). Different concentration of drug (1-1000 ng/mL), either as paclitaxel solution, unconjugated paclitaxel-encapsulated nanoparticles, or paclitaxel-encapsulated transferrin-conjugated-nanoparticles, were added to separate wells. Medium and control nanoparticles (without drug) were used as controls for drug in solution or drug-loaded nanoparticles. The medium was changed on day 2 and on every alternate day thereafter, and no further dose of the drug was added. In a dose-response study, the cell viability was determined at 5 days following treatment. In another set of experiment, cell viability was determined at 2, 5 and 8 days following drug treatment to study the effect of incubation time on drug effect. A standard 3-(4,5-dimethylthiazole-2yl)-2,5-diphenyl tetrazolium bromide (MTT)-based colorimetric assay (CELLTITER 96® AQueous, PROMEGA®, Madison, Wis.) was used to determine cell viability. The reagents were mixed and added to each well (20 μL/well) and the plates were incubated for 3 hours at 37° C. in cell culture incubator. The color intensity was measured at 490 nm using a microplate reader (BT 2000 Microkinetices Reader; BioTek Instruments, Inc., Winooski, Vt.). The effect of drug on inhibition of cell proliferation was calculated as a percentage of cell growth with respect to the respective controls.

### Example 9

### Microscopic Studies

MCF-7 cells were seeded in Bioptechs plates (Bioptechs, Butler, Pa.) at 5×10⁴ cells/plate in 1 mL of growth medium, 24 hours prior to the experiment. To study the intracellular retention of drug, cells were treated either with paclitaxel solution, unconjugated paclitaxel-encapsulated nanoparticles, or paclitaxel-encapsulated transferrin-conjugated-nanoparticles (10 ng/mL) as above. Fluorescently labeled paclitaxel was used for this study. Untreated cells were used as a control to account for autofluorescence. The medium was changed on day 2 and on every alternate day thereafter and no further dose of the drug was added. At various time points, cells were washed three times with PBS before visualization using a confocal microscope equipped with argon-krypton laser (LSM410; Carl Zeiss Microimaging, Thornwood, N.Y.). Excitation of OREGON GREEN® 488 was performed using an argon laser with a wavelength of 488 nm and a long-pass filter of 505 nm.

### Example 10

### Intracellular Drug Levels

MCF-7 cells were plated at a density of 1×10⁵ cells per well per 2 mL in six-well plates and allowed to attach overnight. On the following day, cells were treated with 10 ng/mL tritiated unconjugated paclitaxel-encapsulated nanoparticles, or paclitaxel-encapsulated transferrin-conjugated-nanoparticles, or as a solution. The medium was changed on day 2 and on every other day thereafter, and no further dose of the drug was added. At different time intervals, cells were washed with PBS three times to remove nanoparticles which were not internalized and free drug. Subsequently, cells were lysed by incubation for 30 minutes at 37° C. with 100 μL of 1× cell culture lysis reagent (PROMEGA®, Madison, Wis.). A fraction of each cell lysate (5 μL) was used to determine cell protein levels using Bradford protein assay (BIO-RAD®, Hercules, Calif.) and the remaining portion was lyophilized. The drug from each lyophilized cell lysates was extracted with dimethyl sulfoxide for 48 hours and the drug levels were measured using a liquid scintillation counter (Packard, Downers Grove, Ill.). A standard plot was prepared using an identical protocol.

### Example 11

### Animal Model

Male 6-8 week old athymic nude mice were purchased from the National Institutes of Health. To establish tumors, 2×10⁶ PC3 cells were suspended in 100 μL of RPMI 1640 medium and injected subcutaneously via a 27G^{1/2} gauge needle into the abdominal region. Tumor nodules were allowed to grow to about 50 mm³ prior to receiving different treatments. For intratumoral injections, animals received a single dose of paclitaxel either as nanoparticles suspension or in CREMOPHOR® EL formulation in 0.1 mL with a 25^{1/2} gauge needle placed in the center of the tumor. Tumor dimensions were measured with a digital caliper at regular time intervals and the tumor volume was calculated using the following formula: [length×(width)²]/2.

### Example 12

### Paclitaxel-Loaded, ⁵⁹Fe- and ¹²⁵I-Transferrin-Conjugated Nanoparticles

Paclitaxel-loaded nanoparticles are formulated as described herein. Approximately one-third of transferrin iron-binding pockets can be filled with iron, thus based on the 2.9% weight transferrin/nanoparticle, about 0.1 weight percent iron can be bound to transferrin. Transferrin binds iron avidly with a dissociation constant of approximately 10²² M⁻¹ (Aisen and Listowsky (1980) Annu. Rev. Biochem 49:357-93). Ferric iron couples to transferrin only in the company of an anion (usually carbonate) that serves as a bridging ligand between metal and protein, excluding water from two coordination sites. Paclitaxel-encapsulated, po-transferrin-conjugated nanoparticles are mixed with radioactive iron (⁵⁹Fe/Cl₃; Amersham International) in sodium carbonate buffer, pH 5.9, for 1.5 hours (2% Fe of the transferrin weight is mixed, which for 100 mg nanoparticles, based on bound transferrin, would be 60 μg ⁵⁹Fe/Cl₃). The pH of the suspension is raised to 8.5 with sodium carbonate and mixed for an additional 1.5 to 2 hours. The formulation is dialyzed against water over several hours to remove the buffer salts and unbound iron. Radioactivity in the dialyzed water is monitored to ensure complete removal of free iron. The nanoparticle suspension is lyophilized over two days.

Radioiodination of transferrin is carried out using a standard iodogen method (Lopes, et al. (1993) Cancer Chemother. Pharmacol. 32:235-42). Dual labeling, i.e., ⁵⁹Fe and ¹²⁵I, of transferrin allows for the differentiation of the fate of transferrin and the radiometal. When ⁵⁹Fe-transferrin is internalized within an endocytotic vesicle by receptor-mediated endocytosis, ⁵⁹Fe is released from the protein by a decrease in endosomal pH and passes to its cellular targets. To monitor efflux of nanoparticles from the injection site and the potential for lymphatic drainage, a tracer independent of the iron transport is used, i.e., ¹²⁵I. Dual labeling also allows for the monitoring of radiometal redistribution to normal tissues.

The efficiency of binding of iron to nanoparticles is calculated from the amount of iron that did not bind to nanoparticles. Mean residence times in organs and tumors is calculated from uptake measurements.

Paclitaxel-encapsulated, transferrin-conjugated nanoparticles with and without ⁵⁹Fe or ⁹⁰Y (9.25 Gy or less/mouse) are used to monitor tumor growth to demonstrate effective tumor growth inhibition with nanoparticle drug/radionuclide combinations. A single-dose of nanoparticles (three doses—24 mg and 2.5 mg, 0.25 mg/kg drug equivalent nanoparticles) is injected intratumorally in 25 μL of saline and tumor growth and animal weight and survival are monitored. Tumor dimensions are measured with a digital caliper at regular time intervals and the tumor volume is calculated using the formula: π×(d_{longer})²×(dₛₕₒᵣₜₑᵣ)/6. Controls include ⁵⁹Fe- or ⁹⁰Y-labeled transferrin nanoparticles without paclitaxel; paclitaxel-encapsulated, transferrin-conjugated nanoparticles without radioisotope but with unlabeled Fe; paclitaxel in CREMOPHOR®, and saline.

### Example 13

### Statistical Analysis

Statistical analyses were performed using a Student's t-test. The differences were considered significant for p values of <0.05.

1. A composition for sustained release of a therapeutic agent, said composition comprising a therapeutic agent incorporated in a nanoparticle comprising:
at least one biodegradable polymer,
a functional group, and
transferrin ligand conjugated to the functional group, said therapeutic agent being selected from the group consisting of anti-oxidants, antibiotics, antivirals, fungicides, scabicides, pediculicides, antiseptics, steroids, therapeutic polypeptides, analgesics, anti-inflammatory agents, cancer therapeutic agents, narcotics, local anesthetics, antiangiogenic agents, neuroprotective agents, polysaccharides, vaccines, antigens incorporating the therapeutic agent, and said transferrin ligand being labeled with a beta-emitting radionuclide selected from the group consisting of 59Fe, 90Y, 177Lu, 186Re and 188Re.

2. (canceled)

3. A method for increasing the efficacy of a therapeutic agent comprising administering a composition of claim 1 to a subject in need of treatment with the therapeutic agent, thereby increasing the efficacy of the therapeutic agent in the subject.

4. A method for treating cancer comprising administering to a subject having or at risk of having cancer an effective amount of a cancer therapeutic agent incorporated in a biodegradable nanoparticle, wherein said nanoparticle has transferrin ligand conjugated to the surface thereof and said transferrin ligand is labeled with a beta-emitting radionuclide selected from the group consisting of 59Fe, 90Y, 177Lu, 186Re and 188Re, thereby treating the cancer in the subject.

5. The composition of claim 1, wherein said biodegradable polymer is selected from the group consisting of poly(lactide-co-glycolides), poly(lactic acid), poly(alkylene glycol), polybutylcyanoacrylate, poly(methylmethacrylate-co-methacrylic acid), polyallylamine, polyanhydride, polyhydroxybutyric acid, and polyorthoesters, or a combination of said polymers.

6. The composition of claim 1, wherein said therapeutic agent is a cancer therapeutic agent selected from the group consisting of paclitaxel, mechliorethamine, cyclophosphamide, fluorouracil, thioguanine, carmustine, lomustine, melphalan, chlorambucil, streptozocin, methotrexate, vincristine, bleomycin, vinblastine, vindesine, dactinomycin, daunorubicin, doxorubicin and tamoxifen.

7. The method of claim 4, wherein said therapeutic agent is a cancer therapeutic agent selected from the group consisting of paclitaxel, mechliorethamine, cyclophosphamide, fluorouracil, thioguanine, carmustine, lomustine, melphalan, chlorambucil, streptozocin, methotrexate, vincristine, bleomycin, vinblastine, vindesine, dactinomycin, daunorubicin, doxorubicin and tamoxifen.

8. (canceled)

9. The method of claim 4, wherein said radionuclide produces an anti-tumor effect.

10. The composition of claim 1, wherein said beta-emitting radionuclide is 59Fe.

11. The method of claim 4, wherein said beta-emitting radionuclide is 59Fe.
